(19) United States
(12) Patent Application Publication
(10) Pub. No.: US 2025/0360343 A1
(43) Pub. Date: Nov. 27, 2025

(54) ADJUSTABLE TRANSCRANIAL ULTRASOUND HEADSET

(71) Applicant: Sanmai Technologies, PBC, Sunnyvale, CA (US)

(72) Inventors: Jon Christopher LAKE, Sunnyvale, CA (US); Quentin Owen TODD, Foster City, CA (US); Weijun WANG, Menlo Park, CA (US); Joseph SANGUINETTI, Tucson, AZ (US); Christopher DAFT, Tucson, AZ (US); Stephanie FRENCH, Sunnyvale, CA (US)

(21) Appl. No.: 19/195,150

(22) Filed: Apr. 30, 2025

Related U.S. Application Data
(60) Provisional application No. 63/650,236, filed on May 21, 2024.

(51) Int. Cl. A61N 7/00

(52) U.S. Cl. CPC A61N 7/00; A61N 2007/0026; A61N 2007/0078

(57) ABSTRACT

A transcranial ultrasound headset can hold one or more transducer probe assemblies. The headset can be adjusted to match various human head sizes to securely place the ultrasound probe assemblies on the target areas of a patient's head. In an embodiment, transducer probe assemblies can be placed over a patient's left and right temporal areas, and a third transducer can be placed over the patient's inion. Design techniques and construction materials ensure that transducer probe assemblies are held securely during a transcranial ultrasound session while enhancing patient comfort. An adjustable headband strap around the head is attached to a forehead assembly. The headband strap supports one or more rings. Rings hold ultrasound probe assemblies. The forehead assembly supports pre-built settings for quick size adjustment, allowing rings to be positioned over target areas. An adjustable top strap and chin strap provide additional stability and size customization.

### CROSS-REFERENCE TO RELATED APPLICATIONS

This application claims priority benefit to U.S. Provisional Patent Application No. 63/650,236, entitled “ADJUSTABLE TRANSCRANIAL ULTRASOUND HEADSET”, filed May 21, 2024, the contents of which are incorporated by reference herein in its entirety.

### TECHNICAL FIELD

This invention relates to transcranial ultrasound (TUS) apparatus, specifically an adjustable headset.

### BACKGROUND

Transcranial ultrasound is a non-invasive neuromodulation technique that can be used for various applications, including neurological and psychiatric diseases. Several factors complicate the ultrasound (US) delivery to the anatomical target within the patient's head. For TUS to work effectively, one or more transducers or transducer probes must securely be attached to the patient's head. The probe must be placed close to the scalp to avoid air bubbles. This is complicated by the variations in head measurements and geometry across individuals, especially between males and females. Before the start of the ultrasound session, the position and pose of the probes are adjusted (setup) and verified to ensure effective US delivery to the correct anatomical regions. The probe must remain in place despite patient movements during the TUS session. It is critical to avoid excessive setup time for the TUS procedures, to keep the cost economical by reducing the total treatment time. The TUS treatment device must be comfortable to wear, given that a TUS session can last for up to forty minutes or more. Patients are often sensitive (especially on the head) to the distribution of weight and other sensations caused by the structures in contact with the head. This is an issue if the weight of the TUS device is not balanced—for example, when using a single probe. The patient feels that the head is being weighed excessively in the direction where the probe is placed. This can cause discomfort or pain, given the length of the session. There is a need for a TUS treatment device that patients can wear that can be adjusted to their head structures. The device must allow patients to wear the device securely and comfortably throughout the TUS treatment session. Once a patient's setup is complete, no adjustments should be required during the session and, ideally, setup is repeatable between non-contiguous sessions.

### BRIEF DESCRIPTION OF THE DRAWINGS

FIGS. 1A and 1B show an adjustable transcranial ultrasound headset 100. FIG. 1A shows the headset's front and side view, and FIG. 1B shows its back view.

FIGS. 2A and 2B illustrate the width and length measurements of headset 100.

FIG. 3 illustrates the operation of the forehead assembly 140 in the adjustable headset 100.

FIG. 4 illustrates the operation of the latch assembly 130 and back ring 125.

FIGS. 5A, 5B, 5C, and 5D illustrate ring 110 inside the adjustable headset 100.

FIGS. 6A and 6B show counterweight 120 used to balance the headset 100's weight when a single probe is used.

FIGS. 7A, 7B, and 7C illustrate fiducial 160.

FIG. 8 illustrates the snap-in & out attachment 195 used for the chin strap 195 and top strap 150.

FIGS. 9A and 9B illustrate the use of hook-and-loop fasteners in the top strap 150 and chin strap 195, respectively.

FIG. 10 shows a coupling interface 1000 for the ultrasound probe 170.

### DETAILED DESCRIPTION OF THE INVENTION

A transcranial ultrasound headset can hold one or more transducer probe assemblies. The headset can be adjusted to match various human head sizes to securely place the ultrasound probe assemblies on the target areas of a patient's head. In an embodiment, transducer probe assemblies can be placed over a patient's left and right temporal areas, and a third transducer can be placed over the patient's inion. Design techniques and construction materials ensure that transducer probe assemblies are held securely during a transcranial ultrasound session while enhancing patient comfort. An adjustable headband strap around the head is attached to a forehead assembly. The headband strap supports one or more rings. Rings hold ultrasound probe assemblies. The forehead assembly supports pre-built settings for quick size adjustment, allowing rings to be positioned over target areas. An adjustable top strap and chin strap provide additional stability and size customization.

Attaching an ultrasound probe to a patient's head for transcranial neuromodulation is difficult for several reasons. These problems motivate the innovations disclosed in the present application.

First, the efficacy of a device that cannot steer its ultrasound beam in response to patient motion depends critically on the stability of the probe's location with respect to the head. Motion either laterally across the head or as an angular displacement can be destructive to the effectiveness of the treatment. A high level of probe stability is required for clinical success.

Second, experience shows patients' sensitivity to structures in contact with the head. This constrains the type of headset which can support a transducer during transcranial ultrasound treatments. Patients are sensitive to the weight of the headset and its probe, how it rests on their head, the materials that comprise the areas of patient contact with their skin, and other factors.

Third, it is difficult to reduce the weight of a traditional bulk PZT probe below approximately 100 g, complicating the headset design. This weight constraint is a consequence of several factors. The diameter of the probe needs to be large enough, for example, 70 mm, to adequately focus ultrasound at a frequency low enough to penetrate the skull without large attenuation or beam aberration. The thickness of the piezoelectric material is constrained by its resonant frequency to, for example, around 4 mm for a 500 kHz resonance. Since the density of typical lead zirconate titanate (PZT) is around 7600 kg·m3, small material volumes result in significant weight. Matching layers, an acoustic lens, and the probe case add to the overall weight. Epoxy backing material coating the back side of the transducer stack can be quite thick and is typically doped with tungsten, resulting in a high material density.

Human subjects are always in motion, even if the patient is asked to restrict their movements during the procedure. The length of the procedure is determined by medical needs but can easily be 10-40 minutes, or more.

Finally, the setup time just before the ultrasonic stimulation is started is critical for the system operator and the patient. The headset must be able to be put on easily, and once it is in place, the probe must be moved to the correct treatment location and fixed in place. Once fixed, it should press against the head with enough force to exclude air from the path of the ultrasound as it travels from the probe to the scalp. However, the force must not be large enough to create discomfort for the patient.

The purpose of this application is to disclose a headset design capable of fulfilling this diverse set of requirements.

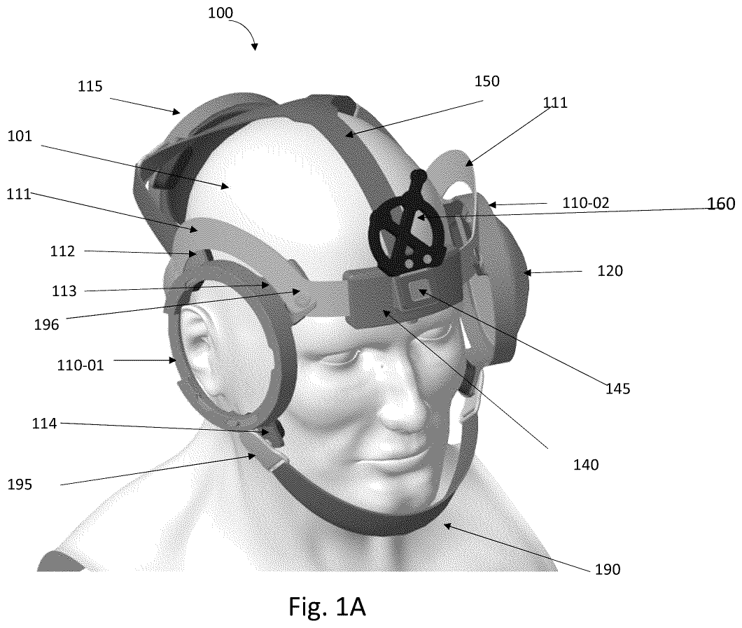

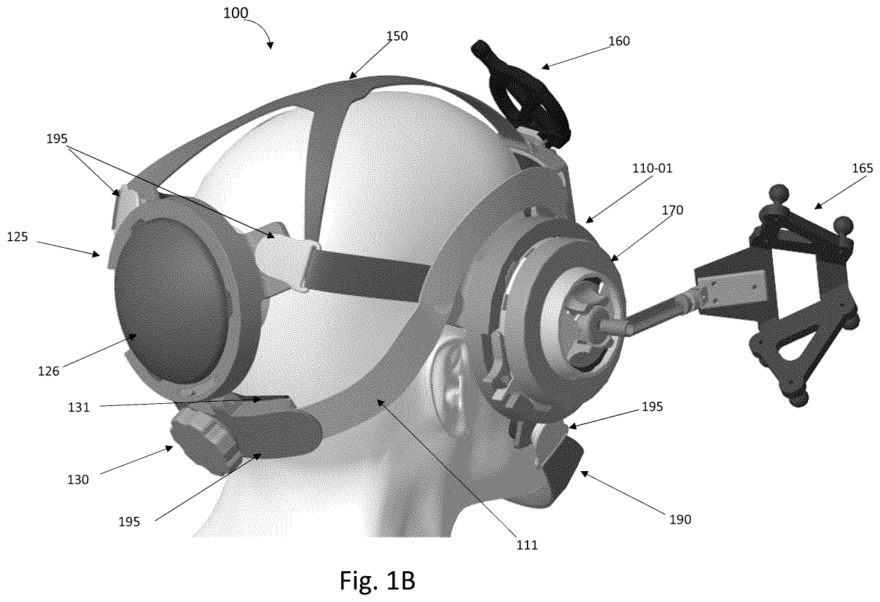

FIGS. 1A and 1B show an adjustable transcranial ultrasound headset 100. FIG. 1A shows the headset's front and side view, and FIG. 1B shows its back view. The Headset 100 can hold one or more transducer probe assemblies and is placed on the patient's head 101. Headset 100 has multiple features that allow it to be adjusted for the patient's head size, and allows transducer assemblies to be firmly attached to a patient's head during treatment. Referring to FIG. 1A, headset 100 includes an adjustable top strap 150 (or overhead strap) that gives a technician visible guidance for placing or setting the headset. Headset 100 includes an adjustable Headband strap 111, which runs around the patient's head 101. It is made of a material tough enough to provide support, allowing it to be strong enough to hold one or more rings (110-01 and 110-02, ultrasound transducer probe holders) while being flexible to conform to the patient's head 101. In an implementation, headband strap 111 is constructed out of PP (Polypropylene). Rings 110 hold the transducer or transducer probe assemblies (not shown in FIG. 1A) or counterweight 120. A forehead assembly 140 attaches to headband strap 111. Forehead assembly 140 can be adjusted to keep the rings 110 within a reasonable range of the patient's temporal area. Other implementations are possible where the ring(s) can be placed at other sites on the patient's head 101. Forehead assembly 140 has pre-built settings that match various human head sizes. In an implementation, forehead assembly 140 has three settings to match the head sizes of 5% Female, 50% Male, and 95% Male head sizes. Top strap 150 connects to forehead assembly 140. Forehead assembly 140 that contacts the patient's skin is constructed to include pads that enhance the patient's comfort using flexible and foamy material such as polyurethane, polyurethane leatherette, etc.

Rings or probe holders 110-01 and 100-02 have three feet 112, 113, and 114, defining a datum on the patient's head 101. Rings 110-01 and 110-02 on the sides of the patient's head 101 provide headset 100 with a stable structure. The three feet 112, 113, and 114 are in contact with the patient's skin, so they are constructed to include pads made of compressible and foamy material to enhance the patient's comfort. Headband strap 111 is attached to rings 110-01 and 110-02 at feet 112 and 113. An adjustable chin strap 190 attaches to rings 110 at foot 114. Chin strap 190 provides overall stability to headset 100 and prevents the headset from being pulled up when top strap 150 is tightened. Chip strap 190 and top strap 150 can be adjusted using a hook and loop fastener (like VELCRO®) or other adjusting mechanisms. Chin strap 190 connects to the feet using a quick-release mechanism 195, a snap-in & out type for quick attachment and release. The diagram illustrates the feet, etc., on the left side only corresponding to ring 110-01. The construction and placement are identical for ring 110-02 on the right side. Headband strap 111 connects to the feet 112 and 113 using hooks 196 or other appropriate mechanism. Forehead assembly 140 includes a quick-release attachment 145 that allows for attaching a head fiducial 160. Fiducial 160 can be used for neuronavigation. The orientation of the fiducial can be flipped so that it can be used with a neuronavigation system placed behind the patient.

Referring to FIG. 1B, headset 100 includes a back ring assembly (or third ring assembly) consisting of a ring (or probe holder) 125 with a latch assembly 130. Back ring 125 can optionally hold a transducer assembly (not shown) and is placed above the patient's inion. This placement can help target the patient's PCC (posterior cingulate cortex). In the figure, a ring cover 126 covers the ring 125. Headband strap 111 attaches to latch assembly 130 using hooks 196 (not shown) or other appropriate methods. The latch assembly 130 is self-locking and tightens the headband strap 111.

Back ring 125 has two feet in contact with the patient's head 101. In the figure, only foot 116 is shown (the other foot is on the left side and is symmetrical). The latch assembly 130 includes a foot 131 in contact with the patient's head. These feet are constructed to include pads made of flexible and foamy material to enhance the patient's comfort. The top strap 150 connects to the back ring 125 feet 116 via a quick-release snap-in & out attachment 195. The top strap 150 connects to the side ring 110 feet 111 (correspondingly to the left side). The top strap 150 tightens the back ring 125 (3^{rd} ring) (using a hook and loop fastener or other adjusting mechanisms).

FIG. 1B shows ring 110-01 holding an ultrasound probe 170. Attached to probe 170 is a probe fiducial 165. Probe fiducial 165 has six balls (not marked) that help a neuronavigation system position the probe 170.

The rings 110-01, 110-02, and 125 act as stable ultrasound transducer probe holders. The probe's position relative to the rings is adjustable to ensure the ultrasound procedure's efficacy. The operation and details of the probe are described in a co-pending application.

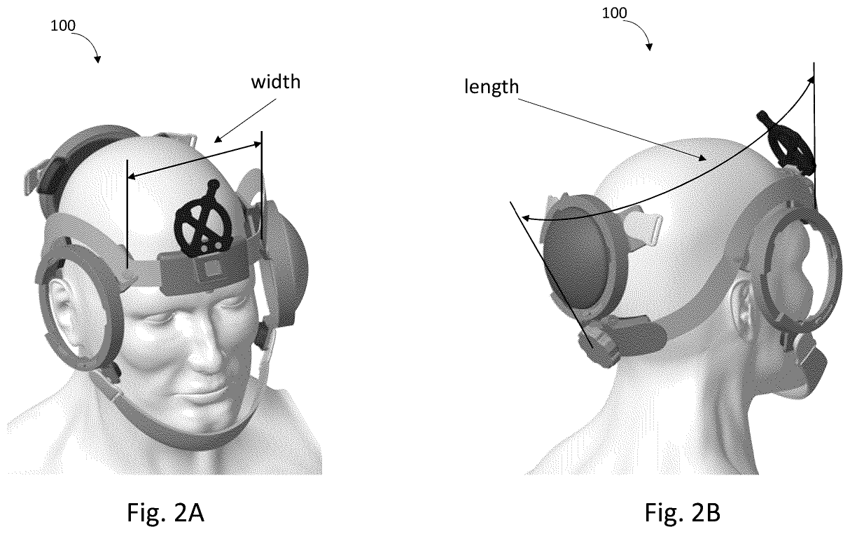

FIGS. 2A and 2B illustrate the width and length measurements of headset 100. Headset 100 has four adjustable features. Forehead assembly 140 has pre-built settings that match various human head sizes for small, medium, and large. The headband strap 111 is flexible. The latch assembly 130 tightens headband strap 111 securely. Forehead assembly 140 and latch assembly 130 adjustments ensure that the rings 110-01 and 110-02 stay inside the temporal target areas for various head sizes. The adjustable top strap 150 uses a hook and loop fastener and tightens the back ring 125. The adjustable Chin strap 190 uses a hook and loop fastener that prevents the headset 100 from being pulled up and helps stabilize the headset 100. FIG. 2A illustrates the width measurement, and FIG. 2B illustrates the length of the adjustable headset 100. In an implementation, the width of the headset can be adjusted between 117 mm and 177 mm, while the length can be adjusted between 212 mm and 312 mm. In the implementation where the forehead assembly 140 has three settings, the width of the headset in the middle position is 147 mm. Once the headset is adjusted to fit a patient's head securely and comfortably, the headset is stable despite movements from the patient throughout the TUS session.

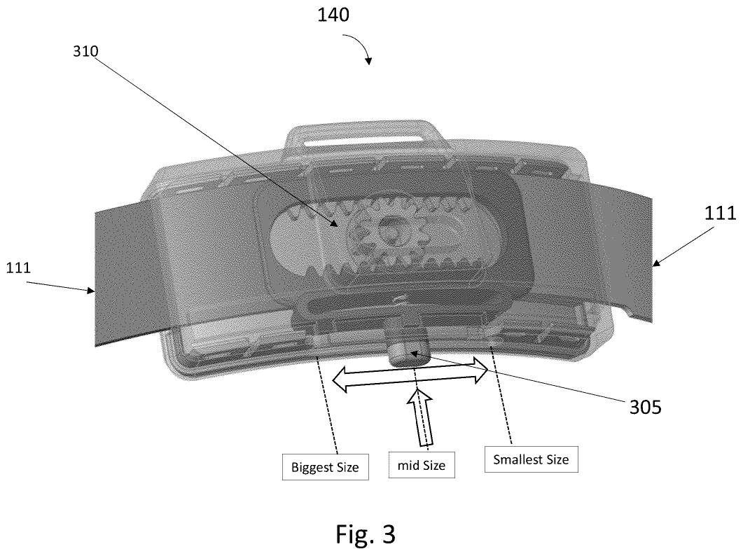

FIG. 3 illustrates the operation of the forehead assembly 140 in the adjustable headset 100. Forehead assembly can adjust the headset's width so that rings 110-01 and 110-02 are placed over temporal target areas for various head sizes. The figure illustrates that three different head sizes can be selected, including gearing mechanism 310, which facilitates this. To change the head size, the button or knob 305 is pushed towards assembly 140, and one of the three sizes is selected by moving the knob 305. In an implementation, the biggest size corresponds to 95% Male head size; the mid-size corresponds to 50% Male, and the smallest size corresponds to 5% Female head size.

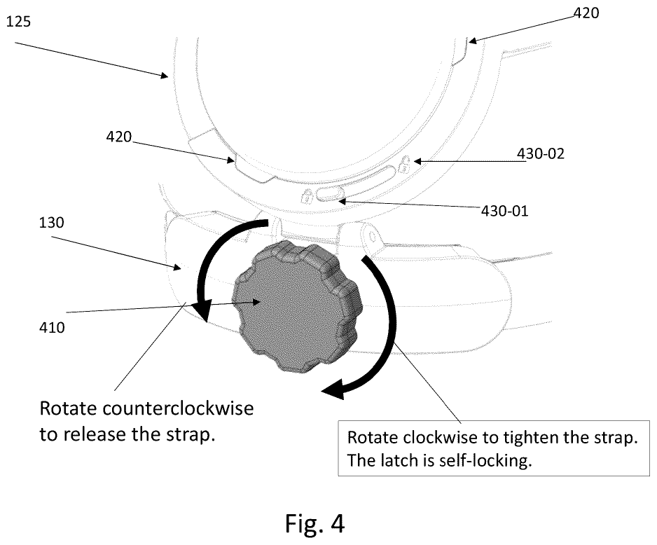

FIG. 4 illustrates the operation of the latch assembly 130 inside the adjustable headset 100. Knob 410 in the latch assembly 130 can be used to tighten or release the headband strap 111. In an implementation, a self-locking with a ramp release pinion mechanism is used in the latch assembly. As the user turns the knob, the headband strap 111 sizes up or down (depending on direction, clockwise to tighten and size down). FIG. 4 also illustrates the attachment of probes or probe covers with ring 125. The ring 125 has recesses 420 to engage with the corresponding tabs or feet of the ultrasound probes or ring cover 126. A slider, when unlocked 430-02, allows the attachment of probe 170 or probe cover 126.

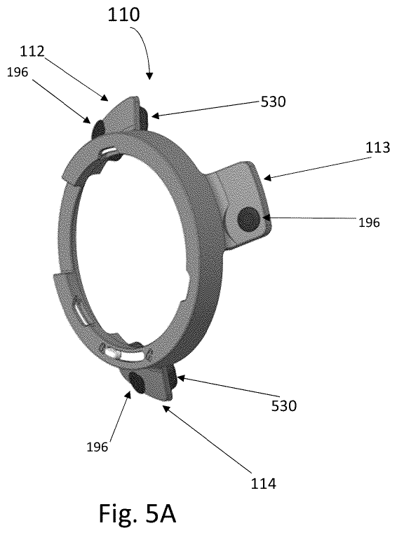

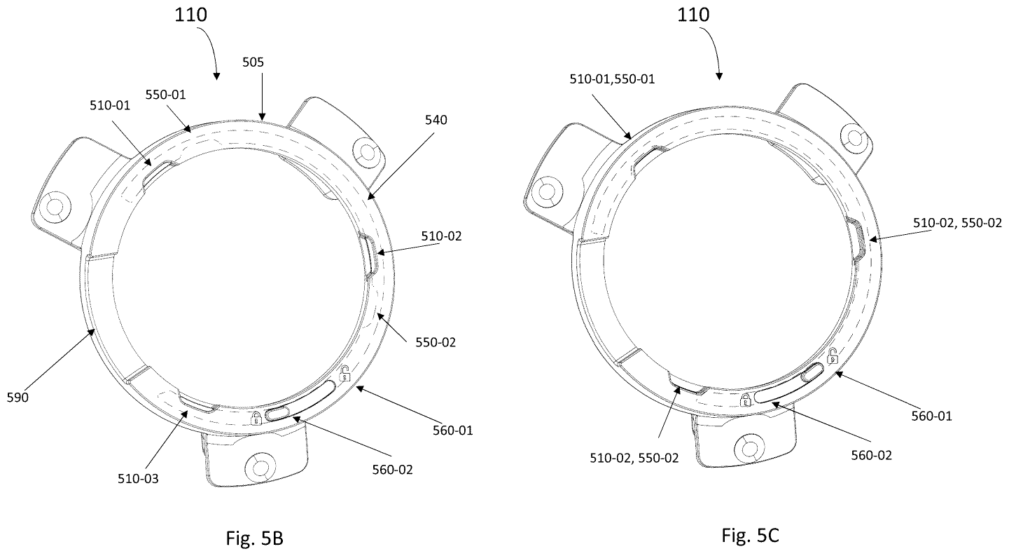

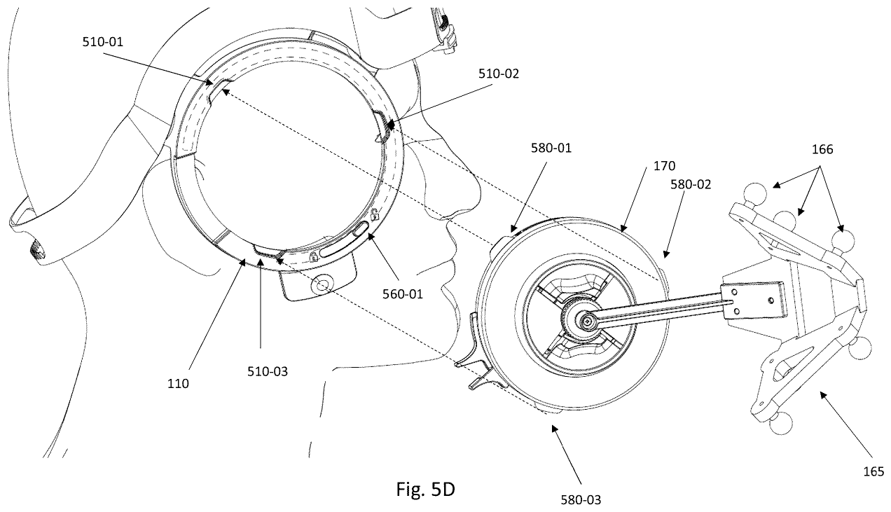

FIG. 5A illustrates ring 110 inside the adjustable headset 100. Special pads 530 underneath feet 112, 113, and 114 are in contact with the patient's skin and constructed out of flexible and foamy materials such as polyurethane, polyurethane leatherette, etc. The pad underneath feet 113 is not visible in this figure. The figure also illustrates hooks 196 used to attach head strap 111 to the feet.

FIGS. 5B and 5C illustrate the operation of the ring 110 for attaching ultrasound probes or counterweights. Ring 110 includes a retainer ring 505 with notched recess pockets 510. Retainer ring 505 includes a sliding latch 540 (shown in the dotted line). The sliding latch 540 can be in an open 560-01 or locked 560-02 position. Sliding latch 540 includes notches 550. When the sliding is open (FIG. 5C), the notches 550 of sliding latch 540 align with notches 510 of retainer ring 505. In the open position, tabs or feet of probe 170 or counterweight 120 can be inserted into the recessed notches of the ring 110. FIG. 5D illustrates the attachment of probe 170 to the ring 110. Tabs 580 of probe 170 are aligned with the notched recesses 510 of ring 110. Sliding latch 540 is unlocked 560-01. Tabs 580 of probe 170 are inserted into notched recesses 510 of ring 110 to attach probe 170 to the headset 100. Sliding lock 540 is moved to the locked 560-02 position to secure the probe. FIG. 5D also illustrates reflecting balls 166 of probe fiducial 165. In the preferred embodiment, probe 170 is attached to ring 110 using three evenly spaced tabs and three notched recesses. Other configurations are possible using different numbers of notches and tabs. Using the same process, a counterweight 120 or probe cover 126 can be attached to rings 110-01, 110-02, or 125. Referring to FIG. 5B, ring 100 illustrates a debossment 590 on the retainer ring 505. The debossment 590 allows for ultrasound probe 170's wires. The debossment 590 allows the ultrasound probe to be smaller while reducing weight. The debossment also reduces the operational complexity.

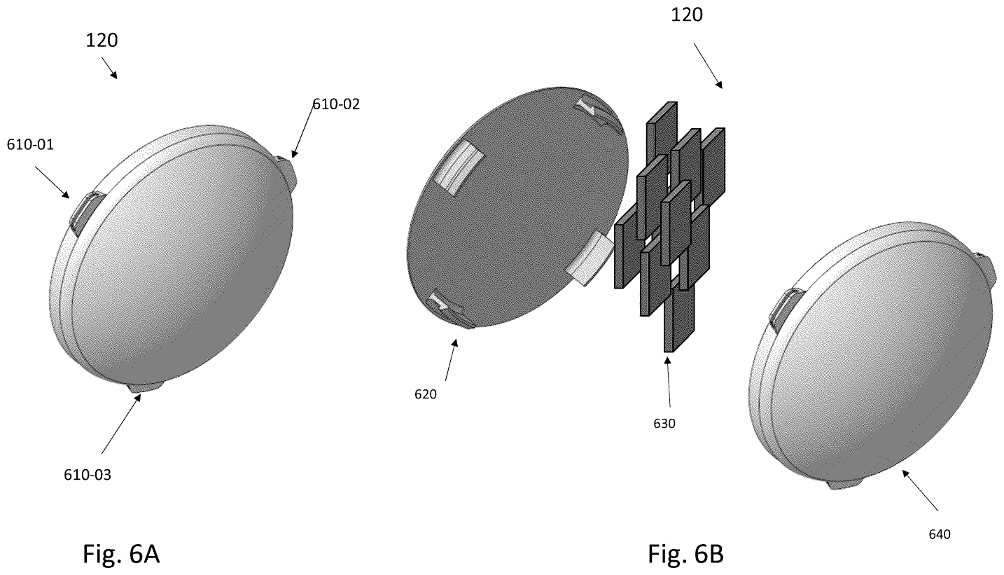

FIGS. 6A and 6B show counterweight 120 used to balance the headset 100's weight when a single probe is used. FIG. 6A shows tabs (or feet) 610-01, 610-02, and 610-03 that engage recesses 510 of the ring 110. FIG. 6B is an exploded view of counterweight 120 and illustrates a counterweight cover 620, weights 630, and counterweight housing 640. Weights 630 can be constructed out of materials such as steel. Weights 630 is selected to match the weight of the ultrasound probe 170. When a single probe is used, the patient's head 101 is weighed down towards the side of the probe. This can cause discomfort or pain to the patient, especially when a TUS session lasts for extended periods. Using a counterweight to balance the weight of the headset 100 can increase the comfort of the patient. Counterweight 120 can be locked into rings 110-01 or 110-02.

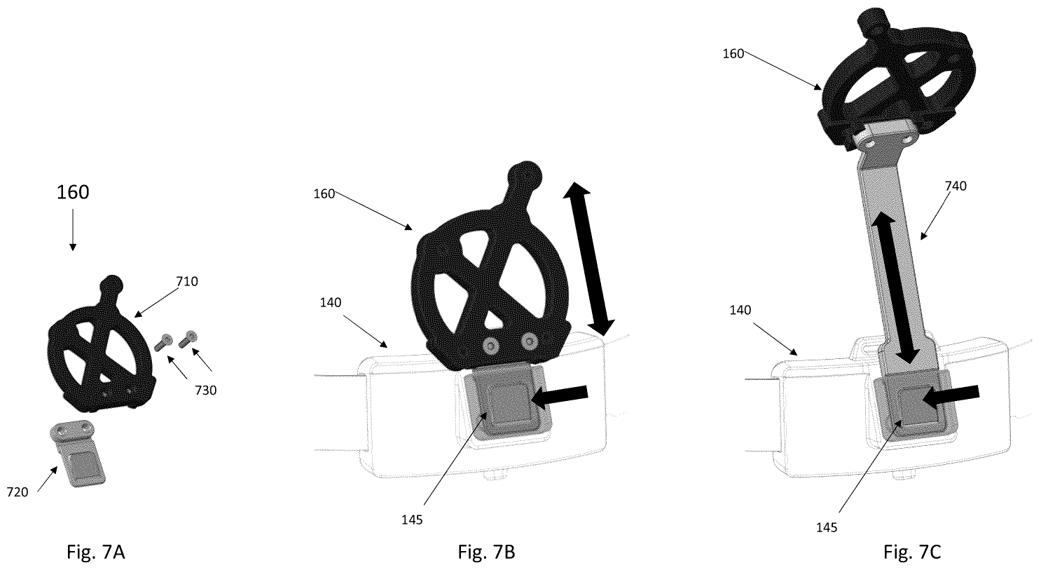

FIGS. 7A, 7B, and 7C illustrate fiducial 160. FIG. 7A shows the major components of the fiducial 160. Fiducial 160 consists of fiducial marker 710 and fiducial attachment 720. Fiducial marker 710 is secured to the attachment 720 using screws 730 or other appropriate methods. FIG. 7B shows the operation of Fiducial 160. Attachment 720 is secured into quick-release attachment 145 inside the forehead assembly 140 by pushing the quick-release attachment 145 towards assembly 140. Similarly, attachment 720 can be removed by pressing on quick-release 145. FIG. 7C shows an extended fiducial attachment 740. The longer fiducial attachment 740 helps improve the line-of-sight of a neuronavigation system. FIG. 7C also illustrates where the fiducial 160 is flipped so that it can be used by a neuronavigation system behind the patient's head 101.

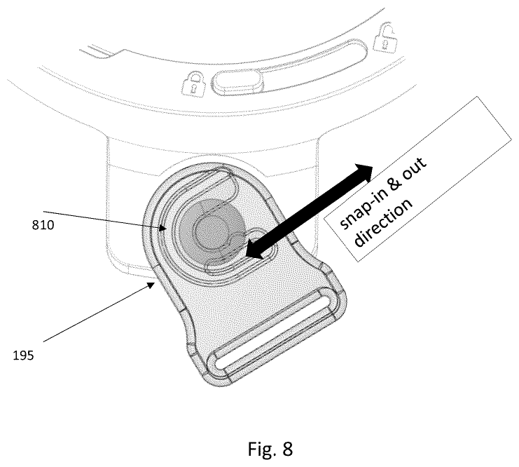

FIG. 8 illustrates the snap-in & out attachment 195 used for the chin strap 195 and top strap 150. The straps have snap attachment 810 that is snapped into the snap-in & out attachment 195.

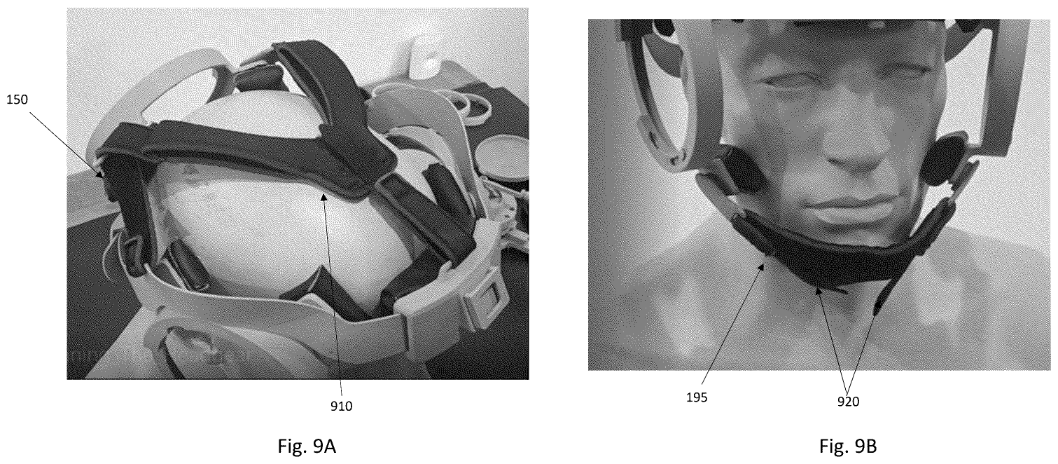

FIGS. 9A and 9B illustrate the use of hook-and-loop fasteners in the top strap 150 and chin strap 195, respectively. The hook-and-loop fasteners allow a clinician to tighten the straps quickly. The chin strap 195 provides overall stability to the headset 100 and prevents the headset from moving up when the top strap 150 is tightened. The top strap 150 is used to secure the back ring 125 in place.

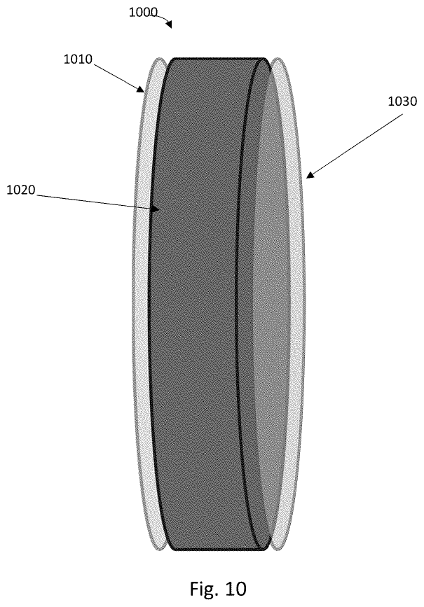

FIG. 10 shows a coupling interface 100 for the ultrasound probe 170. The ultrasound probe 170 needs to meet several requirements. It must be soft and compliant with the contours of the patient's head to exclude air in the ultrasonic path. The surface touching the skin and the surface touching the probe to be gently adhesive so as to further minimize the risk of movement of parts during the treatment. The adhesive for the skin side needs to be biocompatible while the adhesive for the probe side needs to bond well (but temporarily) to the surface of the probe, which may be comprised of silicone. As illustrated in FIG. 10, the coupling interface 1000 is made in three components. A semi-solid gel 1020 provides a conforming interface. Note that in FIG. 10, the three parts are spaced slightly apart for clarity of illustration. In practice, tapes 1010 and 1030 are glued to gel 1020. Item 1010 is a tape, ideally made of a soft polymer, which is very thin compared to the acoustic wavelength. For example, its thickness may be 25-50 microns. It has a biocompatible adhesive on its left-hand face in FIG. 10, and an adhesive specialized for the gel 1020 on its right-hand face. Item 1030 is a similarly thin polymer tape with gel-compatible adhesive on its left-hand face and adhesive specialized for the probe surface on its right-hand face.

1. In some embodiments, an adjustable transcranial ultrasound headset comprises an adjustable top strap, a headband strap comprises a latch assembly for adjustment of the headband strap, a forehead assembly coupled to the adjustable top strap and the headband strap, the forehead assembly comprising one or more settings for headset size adjustment, an adjustable chin strap, and one or more rings for attaching one or more transducer probe assembles, wherein the one or more rings are coupled to the adjustable chin strap and forehead assembly.

2. The adjustable transcranial ultrasound headset of clause 1, wherein the forehead assembly is adjustable between a width of approximately 117 millimeters and 177 millimeters.

3. The adjustable transcranial ultrasound headset of clauses 1 or 2, wherein the forehead assembly is adjustable between a length of approximately 212 millimeters and 312 millimeters.

4. The adjustable transcranial ultrasound headset of any of clauses 1-3, wherein forehead assembly adjusts to reposition the one or more rings over one or more temporal region of a patient.

5. The adjustable transcranial ultrasound headset of any of clauses 1-4, wherein the forehead assembly comprises a plurality of built-in adjustment settings, the plurality of built-in adjustment settings comprising a fifth percentile female head size setting, a fiftieth percentile male head size setting, and a ninety-fifth percentile male head size setting.

6. The adjustable transcranial ultrasound headset of any of clauses 1-5, wherein the headband strap comprises a flexible material.

7. The adjustable transcranial ultrasound headset of any of clauses 1-6, wherein the one or more rings are coupled to the headband strap using one or more hooks.

8. The adjustable transcranial ultrasound headset of any of clauses 1-7, wherein one ring of the one or more rings are coupled to the headband strap so that the one ring is positioned over an inion of a patient, the one or more rings being positioned over a left and right temporal area of a patient.

9. The adjustable transcranial ultrasound headset of any of clauses 1-8, wherein the one or more rings comprise one or more feet with pads positioned to contact skin of a patient.

10. The adjustable transcranial ultrasound headset of any of clauses 1-9, wherein the one or more pads comprise at least one of polyurethane, polyurethane leatherette, foam, or a flexible material.

11. The adjustable transcranial ultrasound headset of any of clauses 1-10, wherein the one or rings comprise a retainer ring, the retainer ring comprising one or more recessed notches for securing the one or more transducer probe assembles.

12. The adjustable transcranial ultrasound headset of any of clauses 1-11, wherein the retainer ring comprises a sliding latch, wherein the sliding latch toggles connection of the one or more recessed notches with the one or more transducer probe assemblies, a counterweight, or a probe cover.

13. The adjustable transcranial ultrasound headset of any of clauses 1-12, wherein the retainer ring comprises a debossment for placement of one or more wires within the retainer ring for attachment to the one or more transducer probe assemblies.

14. The adjustable transcranial ultrasound headset of any of clauses 1-13, wherein one ring of the one or more rings comprise a counterweight and a second ring is coupled to a transducer probe assembly of substantially equivalent weight.

15. The adjustable transcranial ultrasound headset of any of clauses 1-14, wherein the forehead assembly comprises a quick-release attachment for attaching a head fiducial.

16. The adjustable transcranial ultrasound headset of any of clauses 1-15, wherein the head fiducial comprises a fiducial marker and a fiducial attachment.

17. The adjustable transcranial ultrasound headset of any of clauses 1-16, further comprising a probe fiducial coupled to the one or more transducer probe assemblies.

18. The adjustable transcranial ultrasound headset of any of clauses 1-17, wherein the probe fiducial includes one or more reflecting balls.

19. The adjustable transcranial ultrasound headset of any of clauses 1-18, further comprising a back ring assembly comprising a ring and a latch assembly.

20. The adjustable transcranial ultrasound headset of any of clauses 1-19, wherein the back ring assembly is coupled to the one or more transducer probe assemblies.

21. The adjustable transcranial ultrasound headset of any of clauses 1-20, wherein the headband strap is coupled to the back ring assembly via one or more hooks.

22. The adjustable transcranial ultrasound headset of any of clauses 1-21, wherein the top strap is coupled to the back ring assembly via a quick-release snap-fitted attachment.

23. The adjustable transcranial ultrasound headset of any of clauses 1-22, wherein the latch assembly comprises a self-locking ramp release pinion mechanism to tighten the headband strap.

24. The adjustable transcranial ultrasound headset of any of clauses 1-23, further comprising a coupling interface including at least one adhesive tape and a conformable interface layer.

Any and all combinations of any of the claim elements recited in any of the claims and/or any elements described in this application, in any fashion, fall within the contemplated scope of the present invention and protection.

The descriptions of the various embodiments have been presented for purposes of illustration but are not intended to be exhaustive or limited to the embodiments disclosed. Many modifications and variations will be apparent to those of ordinary skill in the art without departing from the scope and spirit of the described embodiments.

Aspects of the present embodiments can be embodied as a system, method or computer program product. Accordingly, aspects of the present disclosure can take the form of an entirely hardware embodiment, an entirely software embodiment (including firmware, resident software, micro-code, etc.) or an embodiment combining software and hardware aspects that can all generally be referred to herein as a “module,” a “system,” or a “computer.” In addition, any hardware and/or software technique, process, function, component, engine, module, or system described in the present disclosure can be implemented as a circuit or set of circuits. Furthermore, aspects of the present disclosure can take the form of a computer program product embodied in one or more computer readable medium having computer readable program code embodied thereon.

Any combination of one or more computer readable medium can be utilized. The computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions can be provided to a processor of a general-purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors can be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams can represent a module, segment, or portion of code, which comprises one or more executable instructions for implementing the specified logical function. It should also be noted that, in some alternative implementations, the functions noted in the block can occur out of the order noted in the figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure can be devised without departing from the basic scope thereof, and the scope thereof is determined by the claims that follow.

What is claimed is:

1. An adjustable transcranial ultrasound headset comprising
an adjustable top strap;
a headband strap comprising a latch assembly for adjustment of the headband strap;
a forehead assembly coupled to the adjustable top strap and the headband strap, the forehead assembly comprising one or more settings for headset size adjustment;
an adjustable chin strap; and
one or more rings for attaching one or more transducer probe assembles, wherein the one or more rings are coupled to the adjustable chin strap and forehead assembly.

2. The adjustable transcranial ultrasound headset of claim 1, wherein the forehead assembly is adjustable between a width of approximately 117 millimeters and 177 millimeters.

3. The adjustable transcranial ultrasound headset of claim 1, wherein the forehead assembly is adjustable between a length of approximately 212 millimeters and 312 millimeters.

4. The adjustable transcranial ultrasound headset of claim 1, wherein forehead assembly adjusts to reposition the one or more rings over one or more temporal region of a patient.

5. The adjustable transcranial ultrasound headset of claim 1, wherein the forehead assembly comprises a plurality of built-in adjustment settings, the plurality of built-in adjustment settings comprising a fifth percentile female head size setting, a fiftieth percentile male head size setting, and a ninety-fifth percentile male head size setting.

6. The adjustable transcranial ultrasound headset of claim 1, wherein the headband strap comprises a flexible material.

7. The adjustable transcranial ultrasound headset of claim 1, wherein the one or more rings are coupled to the headband strap using one or more hooks.

8. The adjustable transcranial ultrasound headset of claim 1, wherein one ring of the one or more rings are coupled to the headband strap so that the one ring is positioned over an inion of a patient, the one or more rings being positioned over a left and right temporal area of a patient.

9. The adjustable transcranial ultrasound headset of claim 1, wherein the one or more rings comprise one or more feet with pads positioned to contact skin of a patient.

10. The adjustable transcranial ultrasound headset of claim 9, wherein the one or more pads comprise at least one of polyurethane, polyurethane leatherette, foam, or a flexible material.

11. The adjustable transcranial ultrasound headset of claim 1, wherein the one or rings comprise a retainer ring, the retainer ring comprising one or more recessed notches for securing the one or more transducer probe assembles.

12. The adjustable transcranial ultrasound headset of claim 11, wherein the retainer ring comprises a sliding latch, wherein the sliding latch toggles connection of the one or more recessed notches with the one or more transducer probe assemblies, a counterweight, or a probe cover.

13. The adjustable transcranial ultrasound headset of claim 11, wherein the retainer ring comprises a debossment for placement of one or more wires within the retainer ring for attachment to the one or more transducer probe assemblies.

14. The adjustable transcranial ultrasound headset of claim 11, wherein one ring of the one or more rings comprise a counterweight and a second ring is coupled to a transducer probe assembly of substantially equivalent weight.

15. The adjustable transcranial ultrasound headset of claim 1, wherein the forehead assembly comprises a quick-release attachment for attaching a head fiducial.

16. The adjustable transcranial ultrasound headset of claim 15, wherein the head fiducial comprises a fiducial marker and a fiducial attachment.

17. The adjustable transcranial ultrasound headset of claim 1, further comprising a probe fiducial coupled to the one or more transducer probe assemblies.

18. The adjustable transcranial ultrasound headset of claim 17, wherein the probe fiducial includes one or more reflecting balls.

19. The adjustable transcranial ultrasound headset of claim 1, further comprising a back ring assembly comprising a ring and a latch assembly.

20. The adjustable transcranial ultrasound headset of claim 19, wherein the back ring assembly is coupled to the one or more transducer probe assemblies.

21. The adjustable transcranial ultrasound headset of claim 19, wherein the headband strap is coupled to the back ring assembly via one or more hooks.

22. The adjustable transcranial ultrasound headset of claim 19, wherein the top strap is coupled to the back ring assembly via a quick-release snap-fitted attachment.

23. The adjustable transcranial ultrasound headset of claim 19, wherein the latch assembly comprises a self-locking ramp release pinion mechanism to tighten the headband strap.

24. The adjustable transcranial ultrasound headset of claim 1, further comprising a coupling interface including at least one adhesive tape and a conformable interface layer.
